(19) United States
(12) Patent Application Publication
(10) Pub. No.: US 2023/0114643 A1
(43) Pub. Date: Apr. 13, 2023

(54) VEHICLE DISPATCH SERVICE SYSTEM

(71) Applicant: TOYOTA JIDOSHA KABUSHIKI KAISHA, Toyota-shi Aichi-ken (JP)

(72) Inventors: Kiyoto Sasaki, Susono-shi Shizuoka-ken (JP); Makoto Kawaharada, Susono-shi Shizuoka-ken (JP); Satoshi Nakamura, Susono-shi Shizuoka-ken (JP); Kenichi Okumura, Gotemba-shi Shizuoka-ken (JP); Naotoshi Kadotani, Sunto-gun Shizuoka-ken (JP)

(73) Assignee: TOYOTA JIDOSHA KABUSHIKI KAISHA, Toyota-shi Aichi-ken (JP)

(21) Appl. No.: 17/945,664

(22) Filed: Sep. 15, 2022

(30) Foreign Application Priority Data
Nov. 9, 2021 (JP) 2021-182302

(51) Int. Cl. G16H 40/20; G01C 21/34

(52) U.S. Cl. CPC G16H 40/20; G01C 21/343

(57) ABSTRACT

A vehicle dispatch service system that provides a vehicle dispatch service in which a vehicle is dispatched to a user to arrive at a hospital at an appointment time of the hospital includes: an arrival time calculation unit that calculates a time at which the vehicle is estimated to arrive at the hospital as an estimated arrival time; an arrival estimation unit that acquires the estimated arrival time from the arrival time calculation unit and estimates whether the user is in time for the appointment time of the hospital; a rebooking unit that resets, when the arrival estimation unit estimates that the user is not in time for the appointment time, a new appointment time as the appointment time based on the estimated arrival time; and rebooking output unit that outputs the new appointment time to a predetermined output destination such that the user recognizes the new appointment time.

### CROSS-REFERENCE TO RELATED APPLICATION

This application claims priority to Japanese Patent Application No. 2021-182302 filed on Nov. 9, 2021, incorporated herein by reference in its entirety.

### BACKGROUND

### 1. Technical Field

The present disclosure relates to a vehicle dispatch service system that provides a vehicle dispatch service for dispatching a vehicle to a user for transportation to and from a hospital.

### 2. Description of Related Art

As this type of system, a vehicle dispatch service system has been proposed that provides a vehicle dispatch service that links a hospital appointment and a reservation of a vehicle to be dispatched to a user in order to make it easier for the elderly and people with disabilities to go to the hospital (refer to Japanese Unexamined Patent Application Publication No. 2021-22334 (JP 2021-22334 A). In the vehicle dispatch service system above, an appointment date and time for consultation are set based on a health condition of the user and information on a doctor, and a vehicle dispatch schedule is created in accordance with the set appointment date and time.

### SUMMARY

However, even when the vehicle is dispatched so as to arrive at the hospital at the appointment time, for example, the vehicle may arrive at the hospital later than the appointment time due to the congestion of the road. However, no consideration is given for such cases in the vehicle dispatch service system above. Therefore, when the vehicle arrives at the hospital later than the appointment time, the waiting list needs to be adjusted again after the user arrives at the hospital and checks in the hospital, which may lead to a situation where the order of consultation is significantly delayed. As described above, in the above-mentioned vehicle dispatch service system, there is a technical issue that the vehicle dispatch service that should be provided in consideration of user convenience cannot sufficiently function.

An object of the present disclosure is to provide a vehicle dispatch service system that can sufficiently function as a vehicle dispatch service provided in consideration of user convenience.

To solve the above issue, an aspect of a vehicle dispatch service system according to the present disclosure is a vehicle dispatch service system that is constructed on a communication network and provides a vehicle dispatch service in which a vehicle is dispatched to a user so as to arrive at a hospital to be in time for an appointment time of the hospital of the user, and includes: an arrival time calculation unit that calculates a time at which the vehicle is estimated to arrive at the hospital as an estimated arrival time; an arrival estimation unit that acquires the estimated arrival time from the arrival time calculation unit and estimates whether the user is in time for the appointment time of the hospital; a rebooking unit that resets, when the arrival estimation unit estimates that the user is not in time for the appointment time, a new appointment time as the appointment time based on the estimated arrival time; and a rebooking output unit that outputs the new appointment time to a predetermined output destination such that the user recognizes the new appointment time.

According to the aspect of the vehicle dispatch service system in the present disclosure, whether the user is in time for the appointment time is estimated based on the estimated arrival time calculated for the vehicle dispatched to the user in accordance with to the appointment time of the hospital. When the user is estimated to be not in time, the new appointment time is reset as the appointment time of the user. The new appointment time is reset based on the estimated arrival time. Therefore, for example, even when the vehicle has to travel significantly later than originally scheduled time due to traffic congestion or the like, the user can arrive at the hospital in time for the reset appointment time. This makes it possible to avoid the situation where, even though the user boards the vehicle so as to be in time for the appointment time, the user is not in time for the appointment time due to unavoidable reasons, and the order of consultation is postponed and the consultation is significantly delayed. As described above, it becomes possible to provide a vehicle dispatch service system that can sufficiently function as a vehicle dispatch service provided in consideration of user convenience.

Such an action and effect according to the present disclosure will be further clarified by the embodiment of the disclosure described below.

### BRIEF DESCRIPTION OF THE DRAWINGS

Features, advantages, and technical and industrial significance of exemplary embodiments of the disclosure will be described below with reference to the accompanying drawings, in which like signs denote like elements, and wherein:

FIG. 1 is a block diagram showing an example of an overall configuration of a vehicle dispatch service system according to the present disclosure;

FIG. 2 is a block diagram showing a hardware configuration of a management server according to the present embodiment;

FIG. 3 is a diagram showing an example of a data structure of data stored in a database according to the present disclosure;

FIG. 4 is a block diagram showing a hardware configuration of a terminal device according to the present embodiment; and

FIG. 5 is a flowchart showing a process executed by the management server according to the present embodiment.

### DETAILED DESCRIPTION OF EMBODIMENTS

### 1. Overall Configuration of Vehicle Dispatch Service System

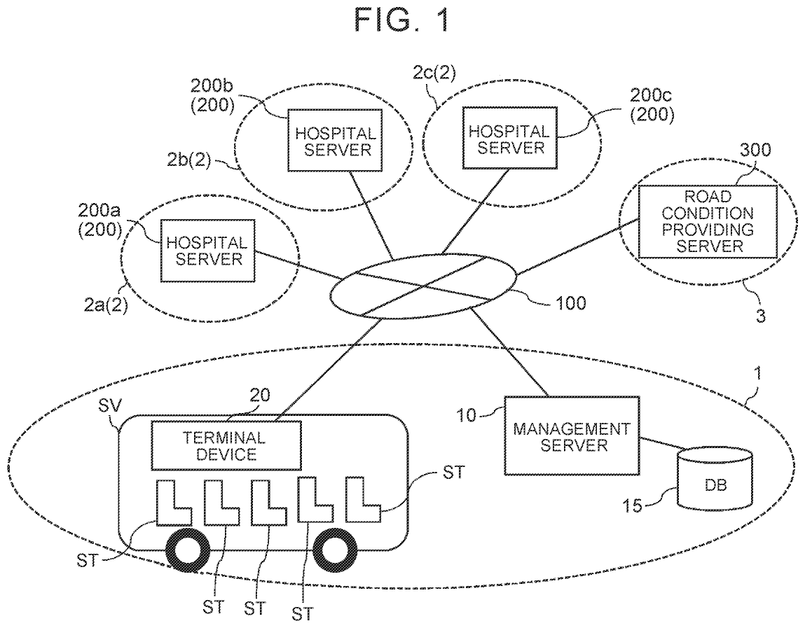

First, with reference to FIG. 1, an example of the overall configuration of a vehicle dispatch service system according to the present disclosure will be described. In a vehicle dispatch service system 1, a vehicle dispatch service is provided in which a service vehicle SV that functions as a Mobility as a Service (MaaS) vehicle is dispatched to a user to be in time for an appointment time for a medical examination (or inspection) at a hospital.

As shown in FIG. 1, the vehicle dispatch service system 1 may include, for example, a management server 10, a database 15 accessible by the management server 10, and a terminal device 20 mounted on the service vehicle SV. The vehicle dispatch service system 1 may be constructed on a communication network, for example, the Internet 100, and the management server 10 and the terminal device 20 may be capable of data communication with each other via the Internet 100.

The service vehicle SV may be provided with a plurality of seats STs for users. The terminal device 20 mounted on the service vehicle SV may have, for example, a function of controlling data communication between the management server 10 and the service vehicle SV and controlling or supporting traveling of the service vehicle SV. The service vehicle SV may be an autonomous vehicle or a chauffeured vehicle. Under the control of an operating organization that operates the vehicle dispatch service provided by the vehicle dispatch service system 1, for example, the management server 10 may be placed in a sales office of the organization or on the cloud. The database 15 stores data necessary for a process executed by the management server 10. The database 15 may be configured to be accessible by the management server 10 via the Internet 100.

Note that, the management server 10 and the database 15 shown in FIG. 1 may have a conceptual configuration, and may be physically realized as one device or may be physically realized by a plurality of devices. For example, the database 15 may be provided in the management server 10. Further, the management server 10 may be realized by the terminal device 20 as the terminal device 20 takes charge of at least a part of the hardware configuration and the process of the management server 10.

As external systems accessible by the management server 10, for example, a hospital system 2a that is a Web-based system that manages an appointment status of a hospital A, a hospital system 2b that is a Web-based system that manages an appointment status of a hospital B, and a hospital system 2c that is a Web-based system that manages an appointment status of a hospital c may be constructed on the Internet 100. Each of the hospital systems 2a, 2b, and 2c may be controlled, for example, by each of hospital servers 200a, 200b, and 200c that can connect to the Internet 100. Hereinafter, when it is not necessary to distinguish each hospital system 2a, 2b, or 2c, the hospital systems 2a to 2c are each referred to as a “hospital system 2”. When it is not necessary to distinguish each hospital server 200a, 200b, or 200c, the hospital servers 200a to 200c are each referred to as a “hospital server 200”. Further, when it is not necessary to distinguish each hospital A, B, or C, the hospitals A to C are each referred to as a “hospital”. Although three hospital systems 2 are shown in FIG. 1, the number of hospital systems 2 accessible by the management server 10 is not limited to this.

Further, a road condition providing system 3 may be constructed on the Internet 100 as an external system accessible by the management server 10. The road condition providing system 3 may be a Web-based system configured to be capable of collecting data on speed information and the like from a plurality of moving vehicles, and providing the road condition of a predetermined range or a designated road (for example, a condition such as congestion, regulation, and/or road closure) as the road condition. The road condition providing system 3 may be controlled by a road condition providing server 300 that can connect to the Internet 100.

In the vehicle dispatch service system 1, a series of processes from setting the appointment time of the hospital to dispatching the service vehicle SV may be the same as the existing vehicle dispatch service linked with the appointment information of the hospital, for example. An example of the series of processes will be described focusing on the processes of the management server 10. For example, the management server 10 compares information on the health condition (or the name of the disease) of the user with information on the doctor to perform matching. Subsequently, the management server 10 causes the user to select a doctor from whom the user wishes to receive a medical examination from among the matched doctors, and accesses the hospital system 2 of the hospital to which the selected doctor belongs and confirms the appointment status of the selected doctor. Then, the management server 10 sets the appointment time of the user based on the appointment status. Further, the management server 10 may create a vehicle dispatch schedule for dispatching the service vehicle SV to the user based on, for example, the address of the user, the location of the hospital with which the appointment is made, the appointment time, the road condition, and the like.

The vehicle dispatch schedule may be created such that at least one user boards one service vehicle SV. For example, the vehicle dispatch schedule may be created for a plurality of users whose appointment times are within a predetermined time and the distances to the hospital with which the appointment is made are within a predetermined range. The vehicle dispatch schedule may include a traveling schedule indicating a travel route or the like on which the service vehicle SV travels. In the traveling schedule, for example, a traveling route for picking up each user and visiting each hospital related to the appointment, an arrival time at a predetermined point on the traveling route, and the like may be set such that each user arrives the hospital in time for the appointment time (for example, 15 minutes before the appointment time). For example, when a user A (not shown) makes an appointment to the hospital A and a user B (not shown) makes an appointment to the hospital B, the service vehicle SV may sequentially pick up each of the user A and the user B in accordance with the traveling schedule and sequentially visit the hospital A and the hospital B so as to send each of the users A and B to the corresponding hospitals A and B, respectively. As described above, the service vehicle SV may pick up each user and visit each hospital so as to be in time for the appointment time in accordance with the traveling schedule. The specific contents set in the vehicle dispatch schedule will be described later. In the present embodiment, a doctor will be described as an example of the target of appointment. However, setting of the appointment time is not limited for the doctor, and may be set for, for example, the clinical department of the hospital or the type of examination.

### 2. Hardware Configuration of Management Server

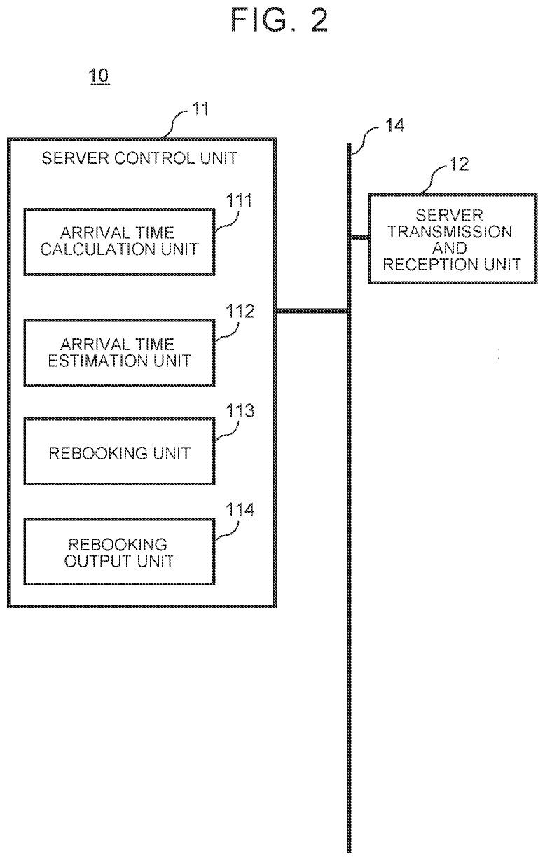

An example of the hardware configuration of the management server 10 will be described with reference to FIG. 2. Since the management server 10 is conceptual as described above, the hardware configuration and its operation shown in FIG. 2 may be realized by, for example, a plurality of server devices. Alternatively, at least a part of the hardware configuration and its operation may be realized by the terminal device 20.

The management server 10 may include, for example, a server control unit 11 and a server transmission and reception unit 12. The server control unit 11 and the server transmission and reception unit 12 are capable of data communication with each other, and may be connected to each other via, for example, a data bus 14. Further, the management server 10 may include, for example, a data input-output unit (not shown) that accepts various operations of a system administrator of the vehicle dispatch service system 1 and outputs various data to the system administrator.

The server transmission and reception unit 12 transmits and receives various data via, for example, the external configuration of the management server 10 (for example, the terminal device 20, the hospital server 200, or the road condition providing server 300) and the Internet 100. The server control unit 11 is configured to control each process executed by the management server 10. The server control unit 11 may be configured as, for example, a computer unit composed of a central processing unit (CPU), and a random access memory (RAM) and a read-only memory (ROM) that are storage areas necessary for the operation of the CPU.

The server control unit 11 may read, for example, a computer program stored in the ROM and execute the program. Further, the server control unit 11 may read, for example, a computer program stored in a computer-readable non-volatile recording medium using a recording medium reading device (not shown). The server control unit 11 may read a computer program from a device (not shown) disposed outside the management server 10 via the Internet 100. The server control unit 11 executes the read computer program. As a result, a logical functional block for executing the operation to be performed by the management server 10 is realized in the server control unit 11. That is, the server control unit 11 can function as a controller for realizing a logical functional block for executing the operation to be performed by the management server 10. FIG. 2 shows an example of the logical functional block realized in the server control unit 11. As shown in FIG. 2, for example, an arrival time calculation unit 111, an arrival estimation unit 112, a rebooking unit 113, and a rebooking output unit 114 may be realized in the server control unit 11.

### 3. Operation of Server Control Unit

The operation of each unit 111 to 114 realized by the server control unit 11 will be described. The transmission and reception of data in respective units 111 to 114 include transmission and reception via the server transmission and reception unit 12.

The arrival time calculation unit 111 may calculate the time at which the service vehicle SV is expected to arrive at the hospital related to appointment of the user as the estimated arrival time. The arrival time calculation unit 111 may calculate the estimated arrival time based on, for example, map information, location information of the hospital, a traveling status of the service vehicle SV, and the road condition (for example, a condition such as congestion, regulation, and/or traffic closure). The arrival time calculation unit 111 may acquire the road condition from, for example, the road condition information provided by the road condition providing system 3.

The arrival estimation unit 112 may acquire the estimated arrival time calculated by the arrival time calculation unit 111 and estimate whether the user is in time for the appointment time of the hospital. For example, the arrival estimation unit 112 may estimate whether the user is in time for the appointment time based on the relationship between the estimated arrival time of the service vehicle SV and the appointment time. The estimation above may take into account, for example, the time required for the user from arrival at the hospital to check-in at the hospital. For example, the arrival estimation unit 112 may estimate that the user is in time for the appointment time when the estimated arrival time is earlier than the appointment time by a predetermined time (for example, 15 minutes) or more, and may estimate that the user is not in time for the appointment time in other cases.

When the arrival estimation unit 112 estimates that the user is not in time for the appointment time, the rebooking unit 113 may reset the appointment time for the user. For example, the rebooking unit 113 may access the hospital server 200 of the hospital related to the appointment, confirm the appointment status of the doctor with whom appointment is made by the user, and set an available appointment time (for example, the time closest to the estimated arrival time) as a new appointment time based on the estimated arrival time. The rebooking unit 113 may reset the appointment time related to the user to the new appointment time.

The rebooking output unit 114 may make an output to, for example, the service vehicle SV (that is, the terminal device 20) that the user is on board in order to notify the user of the reset appointment time (that is, the new appointment time).

### 4. Data Stored in Database

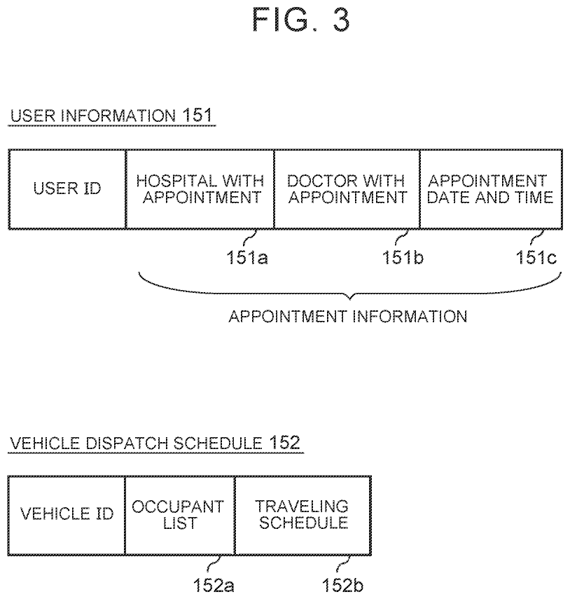

For example, as shown in FIG. 3, the database 15 may store user information 151, a vehicle dispatch schedule 152, and the like. The user information 151 is information on the hospital appointment for each user. The user information 151 may be generated, for example, when the management server 10 sets the appointment time of the hospital for the user. The user information 151 may be composed of, for example, a user identification (ID) for identifying the user and appointment information associated with the user ID. The appointment information may include, for example, appointment hospital information 151a on the hospital related to the appointment, appointment doctor information 151b on the doctor related to the appointment, and appointment date and time 151c indicating the date and time related to the appointment. The appointment hospital information 151a may indicate, for example, a hospital and its location. Note that, each hospital and each doctor may be identified by identification information that identifies each individual.

The vehicle dispatch schedule 152 is information on vehicle dispatch and traveling of the service vehicle SV. In the vehicle dispatch schedule 152, for example, an occupant list 152a and a traveling schedule 152b may be associated with a vehicle ID that identifies each service vehicle SV. The occupant list 152a may show at least one user who is to board the service vehicle SV. In the traveling schedule 152b, as described above, a traveling route in which the service vehicle SV picks up each user and arrives at the hospital related to the appointment in time for the appointment time of the hospital and an estimated arrival time at a predetermined point on the traveling route may be set. The traveling route may indicate, for example, the scheduled travel distance of each road included in the traveling route, the pick-up position of each user (for example, the address of each user), and the location of the hospital. In the traveling schedule 152b, for example, speed information including an average traveling speed of the service vehicle SV corresponding to each road of the traveling route and the like may be set.

### 5. Hardware Configuration of Terminal Device

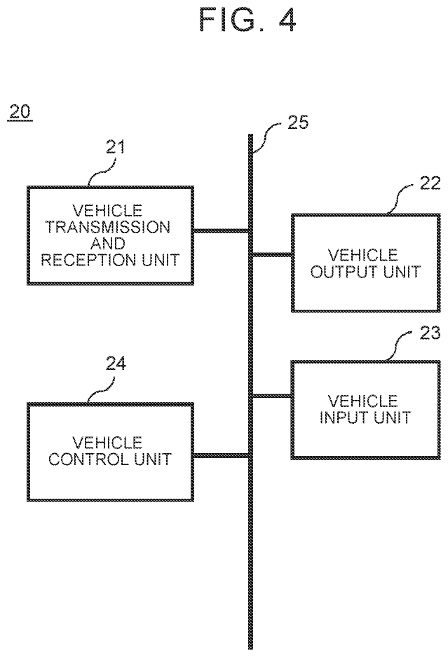

An example of the hardware configuration of the terminal device 20 mounted on the service vehicle SV will be described with reference to FIG. 4. The terminal device 20 may include, for example, a vehicle transmission and reception unit 21, a vehicle output unit 22, a vehicle input unit 23, and a vehicle control unit 24. The vehicle transmission and reception unit 21, the vehicle output unit 22, the vehicle input unit 23, and the vehicle control unit 24 may be connected to each other wirelessly or in a wired manner via, for example, a data bus 25 so as to be capable of data communication. The vehicle transmission and reception unit 21 may be configured to perform transmission and reception of various data to and from the external configuration of the terminal device 20 such as the management server 10 via the Internet 100, for example. The vehicle output unit 22 may be configured to output various data to the user, for example. The data output includes screen output, audio output, and the like. The vehicle input unit 23 may be configured to receive data input of various data by the user, for example. The data input includes, for example, input via various input devices such as a keyboard and a mouse, contact input to a touch panel, buttons, and the like, non-contact input, and voice input.

The vehicle control unit 24 may be configured to control each process in the terminal device 20 by controlling the operations of the other units 21, 22, and 23. The vehicle control unit 24 may be configured as, for example, a computer unit composed of a CPU, and a RAM and a ROM that are storage areas necessary for the operation of the CPU. The vehicle control unit 24 may function as, for example, a user interface of the management server 10. For example, the vehicle ID of the host vehicle may be stored in the ROM. The vehicle control unit 24 may, for example, cause the user to input data in accordance with a command from the management server 10 and transmit the input data to the management server 10. Further, for example, the vehicle control unit 24 may acquire data from the management server 10 and output the acquired data to the user in accordance with a command from the management server 10. The vehicle control unit 24 may be configured to control or support traveling of the service vehicle SV. For example, the vehicle control unit 24 may be configured to be able to specify the position of the host vehicle that is traveling by a predetermined self-positioning technique using electromagnetic induction, the global positioning system (GPS), a three-dimensional map, or the like. The vehicle control unit 24 may be configured to measure the traveling speed of another vehicle, for example, a preceding vehicle and/or a following vehicle, and calculate an average traveling speed within a predetermined time. The vehicle control unit 24 may be configured to realize at least a part of each of the above-mentioned units 111 to 114 of the server control unit 11.

The vehicle output unit 22 and the vehicle input unit 23 may be provided as an input-output set for at least some of the seats ST. That is, the input-output sets composed of the vehicle output units 22 and the vehicle input units 23 may be respectively provided for at least some of the seat STs in the service vehicle SV. With the above, the vehicle output unit 22 and the vehicle input unit 23 function as a seat output unit and a seat input unit, respectively. For example, a set ID for identifying each input-output set by the vehicle control unit 24 may be assigned to each input-output set. For example, when the user ID is input to the vehicle input unit 23 of the input-output set provided in the seat ST in which the user is seated, the vehicle control unit 24 may associate the user ID with the set ID and store association information.

### 6. Process in Vehicle Dispatch Service System

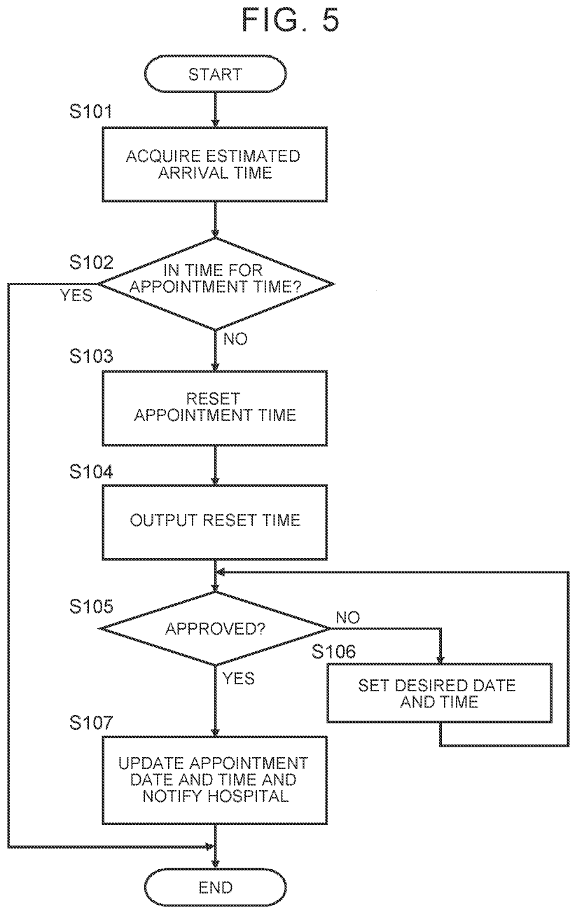

The vehicle dispatch service system 1 according to the present disclosure is configured such that whether the user who is on board the service vehicle SV is in time for the appointment time of the hospital is estimated, and when the user is estimated to be not in time, the appointment time is reset. An example of a process executed by the server control unit 11 of the management server 10 for the resetting will be described in accordance with a processing routine shown in FIG. 5. The server control unit 11 may start the processing routine shown in FIG. 5 for each user who is on board the service vehicle SV that is traveling at, for example, an appropriate timing (for example, periodically at predetermined intervals, or with a predetermined start operation by the user or the driver as a trigger).

The service vehicle SV that is traveling may be specified, for example, based on the traveling schedule 152b of the vehicle dispatch schedule 152, or by the start signal transmitted from the terminal device 20 of the service vehicle SV at the start of traveling. Further, the user who is on board the service vehicle SV may be specified by, for example, the occupant list 152a related to the specified service vehicle SV. Various types of information related to the appointment for each user may be confirmed, for example, by appropriately referring to the appointment information of the user information 151.

When the processing routine is started, the arrival estimation unit 112 of the server control unit 11 may cause the arrival time calculation unit 111 to calculate the estimated arrival time at which the service vehicle SV arrives at the hospital related to the appointment of the user and acquire the estimated arrival time (step S101). When calculating the estimated arrival time, the arrival time calculation unit 111 may acquire, for example, the current position and the traveling status of the service vehicle SV from the terminal device 20, and acquire the road condition information related to the road condition of each road (for example, the condition such as congestion, regulation, and/or road closure) from the road condition providing server 300. The arrival time calculation unit 111 may calculate, for each user, the estimated arrival time that is the time at which the user is estimated to arrive at the hospital related to the appointment based on, for example, the current position of the service vehicle SV on the traveling route, the location of the hospital related to the appointment, the traveling status of the service vehicle SV, the road condition information, and the speed information set in the traveling schedule 152b. The traveling status of the service vehicle SV may include, for example, at least one of the current traveling speed (or average speed) of the host vehicle and the current traveling speed (or average speed) of the preceding vehicle and/or the following vehicle.

Subsequently, the arrival estimation unit 112 may estimate whether each user is in time for the appointment time (step S102). As described above, the arrival estimation unit 112 may make the estimation based on the relationship between the estimated arrival time calculated by the arrival time calculation unit 111 and the appointment time of the user. For example, the arrival estimation unit 112 may estimate that the user is in time for the appointment time when the estimated arrival time is earlier than the appointment time by a predetermined time (for example, 15 minutes) or more, and may estimate that the user is not in time for the appointment time in other cases. When the arrival estimation unit 112 estimates that the user is in time for the appointment time (step S102: Yes), the processing routine that is currently executed may be terminated.

On the other hand, when the arrival estimation unit 112 estimates that the user is not in time for the appointment time (step S102: No), the rebooking unit 113 of the server control unit 11 may reset the appointment time by setting a new appointment time (step S103). For example, the rebooking unit 113 may access the hospital server 200 of the hospital related to the appointment, confirm the appointment status of the doctor related to the appointment, and set a new appointment time based on the appointment status and the estimated arrival time. For example, the rebooking unit 113 may set the time at which appointment is available and that is closest to the estimated arrival time as the new appointment time.

Subsequently, the rebooking output unit 114 of the server control unit 11 may execute a reset time output process of outputting the reset appointment time to a predetermined output destination (step S104). As the reset time output process, the rebooking output unit 114 may output rebooking information including, for example, the new appointment time, the hospital related to the appointment, and the user ID to the service vehicle SV (that is, the terminal device 20) such that the user who is subject to the process is notified of the new appointment time via the vehicle output unit 22. In the terminal device 20 of the service vehicle SV that receives the rebooking information, the vehicle control unit 24 may specify the input-output set associated with the user related to the rebooking information based on the above-mentioned association information, and notify the user of the new appointment time via the vehicle output unit 22 of the specified input-output set. For example, the vehicle control unit 24 may display the hospital related to the appointment and the new appointment time on the screen serving as the vehicle output unit 22 together with a message indicating that the appointment has been reset.

The rebooking output unit 114 may, for example, ask the user whether to approve the displayed new appointment time (step S105). For example, when the user approves the new appointment time by performing a predetermined approval operation with the vehicle input unit 23 of the specified input-output set (step S105: Yes), the server control unit 11 may, for example, update the appointment date and time 151c of the user information 151 to the new appointment time and notify the hospital server 200 of the hospital related to the appointment of the doctor related to the appointment and the new appointment time as the reset appointment time (step S107).

On the other hand, when the user does not approve the new appointment time by, for example, performing a predetermined denial operation with the vehicle input unit 23 (step S105: No), the server control unit 11 may execute a desired date and time setting process (step S106). In the desired date and time setting process, the server control unit 11 may, for example, cause the user to input the desired date and time for appointment as the desired date and time. Then, the server control unit 11 may acquire the appointment status of the doctor related to the appointment from the hospital server 200 of the hospital related to the appointment, set the date and time when the appointment is available and that is closest to the input desired date and time as the new appointment date and time, and return to step S105 to obtain an approval of the user on the new appointment date and time. After that, the server control unit 11 may execute steps S105 to S107 for the new appointment date and time. The processes in steps S105 to S107 for the new appointment date and time may be the same processes as the above-mentioned processes for the new appointment time.

As described above, according to the vehicle dispatch service system in the present disclosure, a vehicle dispatch service allowing the user to make an appointment again while the user is still on board the vehicle even when the dispatched vehicle is not in time for the appointment time of the hospital can be provided.

The arrival time calculation unit 111 may be provided as a functional block generated by the vehicle control unit 24 of the service vehicle SV, and the arrival estimation unit 112 may cause the arrival time calculation unit 111 provided in the service vehicle SV to calculate the estimated arrival time and acquire the calculated estimated arrival time. In this case, the estimated arrival time may be calculated by the same method as when the arrival time calculation unit 111 is provided in the management server 10. The terminal device 20 may be configured to be accessible to the road condition providing server 300. Further, the arrival time calculation unit 111 may use, for example, a non-illustrated external system (hereinafter, referred to as “non-illustrated system”) to calculate the estimated arrival time. The non-illustrated system may be, for example, a Web-based system that can provide an estimated time (or an estimated time until arrival) to arrive at a second position when the vehicle moves from a first point to a second point on the map. In this case, the arrival time calculation unit 111 may provide the non-illustrated system with the current position of the service vehicle SV as the first point and the location of the hospital related to the appointment as the second point and cause the non-illustrated system to calculate the above-mentioned estimated time (or the above-mentioned estimated time until arrival). The arrival time calculation unit 111 may calculate the estimated arrival time according to the present disclosure based on the estimated time (or estimated time until arrival) calculated by the non-illustrated system. The arrival time calculation unit 111 may adopt the estimated time calculated by the non-illustrated system as the estimated arrival time according to the present disclosure. When the road condition providing system 3 has the function of the non-illustrated system, the arrival time calculation unit 111 may use the road condition providing system 3 as the non-illustrated system.

Only one input-output set of the vehicle output unit 22 and the vehicle input unit 23 may be provided at a predetermined position of the service vehicle SV. In this case, for example, the mobile terminal of the user may be notified that the appointment time has been reset. Then, the user who receives the notification may move to a position where the input-output set is provided in order to operate the vehicle input unit 23 and confirm the information output by the vehicle output unit 22. The process after the reset time output process (step S104) may be executed via the vehicle output unit 22 and the vehicle input unit 23 in a manner such that the user who receives the notification inputs the user ID of the user with the vehicle input unit 23, for example.

In the reset time output process, the rebooking output unit 114 of the server control unit 11 may add the rebooking information including the new appointment time to the mobile terminal (smartphone, tablet, portable computer, etc.) in addition to or in place of the service vehicle SV. In this case, for example, the user information 151 may include information for specifying the mobile terminal of the user (for example, an e-mail address, etc.), and the rebooking output unit 114 may refer to the specifying information. In the mobile terminal that receives the new appointment time, the new appointment time may be output so as to be recognizable by the user via the output unit of the mobile terminal. Input and output related to the user in the process relating to the approval of the new appointment time (step S105 and step S106 in FIG. 5) may be performed via the input unit and the output unit of the mobile terminal. The input received by the input unit of the mobile terminal may include, for example, screen input, key input, button input, and voice input. The output provided by the output unit of the mobile terminal may include, for example, screen output and audio output.

### Appendices

The following appendices will be further disclosed with respect to the embodiment described above.

### Appendix 1

A vehicle dispatch service system according to Appendix 1 is a vehicle dispatch service system that is constructed on a communication network and provides a vehicle dispatch service in which a vehicle is dispatched to a user so as to arrive at a hospital to be in time for an appointment time of the hospital of the user, and includes: an arrival time calculation unit that calculates a time at which the vehicle is estimated to arrive at the hospital as an estimated arrival time; an arrival estimation unit that acquires the estimated arrival time from the arrival time calculation unit and estimates whether the user is in time for the appointment time of the hospital; a rebooking unit that resets, when the arrival estimation unit estimates that the user is not in time for the appointment time, a new appointment time as the appointment time based on the estimated arrival time; and a rebooking output unit that outputs the new appointment time to a predetermined output destination such that the user recognizes the new appointment time.

According to the vehicle dispatch service system in Appendix 1, whether the user is in time for the appointment time is estimated based on the estimated arrival time calculated for the vehicle dispatched to the user in accordance with to the appointment time of the hospital. When the user is estimated to be not in time, the new appointment time is reset as the appointment time of the user. The new appointment time is reset based on the estimated arrival time. Therefore, for example, even when the vehicle has to travel significantly later than originally scheduled time due to traffic congestion or the like, the user can arrive at the hospital in time for the reset appointment time. This makes it possible to avoid the situation where, even though the user boards the vehicle so as to be in time for the appointment time, the user is not in time for the appointment time due to unavoidable reasons, and the order of consultation is postponed and the consultation is significantly delayed.

The process by the arrival estimation unit may be executed at an appropriate timing, such as at a predetermined timing or in response to a request from a user or the like. When the process is executed in response to a request of the user, it is possible to eliminate anxiety of the user. When the process is executed periodically or at a timing such as a predetermined time before the original appointment time, a more accurate estimated arrival time can be obtained. As described above, the vehicle dispatch service system in Appendix 1 can sufficiently function as a vehicle dispatch service provided in consideration of user convenience. Note that, the arrival time calculation unit may be provided in the vehicle or in a server that executes processes related to this system. The arrival time calculation unit may be configured to calculate the estimated arrival time based on a preset traveling speed and/or a traveling status of the vehicle. Alternatively, the arrival time calculation unit may be configured to use an external Web-based system capable of providing an estimated required time (or an estimated time to arrive at a destination) based on movement between two points by the vehicle. Use of an external Web-based system makes it possible to reduce the processing load of the vehicle dispatch service system in Appendix 1.

### Appendix 2

The vehicle dispatch service system according to Appendix 2 is the vehicle dispatch service system according to Appendix 1 in which: the vehicle is provided with a plurality of seats; each of at least some of the seats is provided with a seat output unit that outputs predetermined information so as to be recognizable to the user; and the rebooking output unit outputs the new appointment time to the vehicle as the predetermined output destination such that the user recognizes the new appointment time via the seat output unit provided in the seat corresponding to the user.

According to the vehicle dispatch service system in Appendix 2, the user being seated on the seat in the vehicle can confirm the appointment time that is rebooked while being seated in the vehicle. Further, even when the user does not carry a mobile terminal, it is possible to notify the user of the rebooked appointment time. This makes it possible to further enhance the convenience for the user of the vehicle dispatch system in which the appointment time is reset.

### Appendix 3

The vehicle dispatch service system according to Appendix 3 is the vehicle dispatch service system according to Appendix 1 in which the rebooking output unit outputs the new appointment time to a mobile terminal carried by the user as the predetermined output destination such that the user recognizes the new appointment time via the mobile terminal.

According to the vehicle dispatch service system in Appendix 3, the user in the vehicle can confirm the rebooked appointment time via the mobile terminal carried by the user. Notification to the mobile terminal of the user makes it possible to sufficiently protect the privacy related to hospital appointments. This makes it possible to further enhance the convenience for the user of the vehicle dispatch system in which the appointment time is reset.

### Appendix 4

The vehicle dispatch service system according to Appendix 4 is the vehicle dispatch service system according to Appendix 1 in which: the arrival time calculation unit is provided in the vehicle; and the arrival estimation unit causes the arrival time calculation unit provided in the vehicle to calculate the estimated arrival time.

According to the vehicle dispatch service system in Appendix 4, the arrival time calculation unit is provided in the vehicle. Therefore, for example, the processing load of the server of the system can be reduced. Further, when the car navigation system of the vehicle or the like is provided with the arrival time estimation function, the function can be used for the arrival time calculation unit of the present disclosure.

### Appendix 5

The vehicle dispatch service system according to Appendix 5 is the vehicle dispatch service system according to Appendix 1 in which the arrival time calculation unit calculates the estimated arrival time based on a traveling status of the vehicle and road condition information acquired from a predetermined road condition providing system.

According to the vehicle dispatch service system in Appendix 5, the estimated arrival time can be calculated based on the actual traveling status of the vehicle. The traveling status of the vehicle may include, for example, the current traveling speed (or average speed) of the host vehicle, the current traveling speed (or average speed) of the preceding vehicle and/or the following vehicle, and the like. Further, when the road condition providing system that provides road condition information based on traveling speed data and GPS data acquired from a plurality of vehicles that is actually traveling is used, a more accurate estimated arrival time can be calculated.

The present disclosure can be appropriately modified to the extent that the modification does not contradict the gist or idea of the disclosure that can be read from the claims and the entire specification, and the vehicle dispatch service system accompanied by such modification is also included in the technical idea of the present disclosure.

What is claimed is:

1. A vehicle dispatch service system that is constructed on a communication network and provides a vehicle dispatch service in which a vehicle is dispatched to a user so as to arrive at a hospital to be in time for an appointment time of the hospital of the user, the vehicle dispatch service comprising:
an arrival time calculation unit that calculates a time at which the vehicle is estimated to arrive at the hospital as an estimated arrival time;
an arrival estimation unit that acquires the estimated arrival time from the arrival time calculation unit and estimates whether the user is in time for the appointment time of the hospital;
a rebooking unit that resets, when the arrival estimation unit estimates that the user is not in time for the appointment time, a new appointment time as the appointment time based on the estimated arrival time; and
a rebooking output unit that outputs the new appointment time to a predetermined output destination such that the user recognizes the new appointment time.

2. The vehicle dispatch service system according to claim 1, wherein:
the vehicle is provided with a plurality of seats;
each of at least some of the seats is provided with a seat output unit that outputs predetermined information so as to be recognizable to the user; and
the rebooking output unit outputs the new appointment time to the vehicle as the predetermined output destination such that the user recognizes the new appointment time via the seat output unit provided in the seat corresponding to the user.

3. The vehicle dispatch service system according to claim 1, wherein the rebooking output unit outputs the new appointment time to a mobile terminal carried by the user as the predetermined output destination such that the user recognizes the new appointment time via the mobile terminal.

4. The vehicle dispatch service system according to claim 1, wherein:
the arrival time calculation unit is provided in the vehicle; and
the arrival estimation unit causes the arrival time calculation unit provided in the vehicle to calculate the estimated arrival time.

5. The vehicle dispatch service system according to claim 1, wherein the arrival time calculation unit calculates the estimated arrival time based on a traveling status of the vehicle and road condition information acquired from a predetermined road condition providing system.
